(19) United States
(12) Patent Application Publication
(10) Pub. No.: US 2012/0116223 A1
(43) Pub. Date: May 10, 2012

(54) SYSTEM FOR MEASURING AND TRACKING HUMAN BODY FAT

(75) Inventors: Luiz B. DA SILVA, Danville, CA (US); George Yoseung CHOI, Menlo Park, CA (US); Igor G. KOCHEMASOV, Nizhny Novgorod (RU); Drew A. STARK, Livermore, CA (US)

(73) Assignee: INTELAMETRIX, INC., Livermore, CA (US)

(21) Appl. No.: 13/349,976

(22) Filed: Jan. 13, 2012

Related U.S. Application Data
(63) Continuation of application No. 12/464,063, filed on May 11, 2009, now abandoned.
Application No. 12/464,063 is a continuation-in-part of application No. 11/415,560, filed on May 1, 2006, now abandoned.
Application No. 11/415,560 is a continuation-in-part of application No. 11/302,039, filed on Dec. 12, 2005, now abandoned.
(60) Provisional application No. 60/676,325, filed on Apr. 30, 2005.
(60) Provisional application No. 60/634,911, filed on Dec. 10, 2004.

(51) Int. Cl. A61B 8/00

(52) U.S. Cl. 600/442

(57) ABSTRACT

A system for evaluating health, wellness and fitness, and in particular, to a system that uses an ultrasound transducer to accurately measure fat thickness at a plurality of sites on the human body, records these measurements for long term monitoring, and based on the plurality of measurements calculates the total body composition. The system includes a central control unit to analyze the measurement and display the results in a variety of formats.

### CROSS-REFERENCE TO RELATED APPLICATIONS

This application is a continuation of U.S. application Ser. No. 12/464,063 filed May 11, 2009, which is a continuation-in-part of U.S. application Ser. No. 11/415,560, filed May 1, 2006, now abandoned, which claims priority to U.S. Provisional Application No. 60/676,325, filed Apr. 30, 2005, and is a continuation-in-part of U.S. patent application Ser. No. 11/302,039, filed Dec. 12, 2005, now abandoned, which claims priority to U.S. Provisional Application No. 60/634,911, filed Dec. 10, 2004, all of which are incorporated herein by reference.

### BACKGROUND OF THE INVENTION

1. Field of the Invention

The present disclosure generally relates to the fields of fitness, and healthcare, and cosmetic surgery generally. More particularly, the disclosure relates to systems, devices and methods that measure and record fat and muscle thickness at a plurality of sites on the human body with a handheld apparatus utilizing ultrasound. The system can monitor changes in adipose and muscle tissue due to changes in fitness, health, surgery, trauma or disease. The present system and method can also be used to measure total body fat.

2. Description of Related Art

Knowledge of the thickness of tissue layers, and in particular adipose (fat) and muscle tissue, can be important in the evaluation of the fitness and health of an individual. There are a variety of techniques currently used to measure the thickness of the adipose layer. For example skin calipers can be used to measure the thickness of the skin fold produced when the operator pinches a subject's skin. Various equations are used to predict body density and the percent of body adipose tissue (American College of Sports Medicine (ACSM) “Guidelines For Exercise Testing And Prescription”, 53-63 (1995)). However, there are many drawbacks to this form of adipose tissue measurement. These measurements are heavily dependent on the operator, and errors and variations frequently occur. Skin fold calipers can only provide an estimate of tissue thickness and are not particularly accurate for tracking small changes.

Another means of determining body density and estimating percent body adipose tissue is a generalized measurement called hydrostatic weighing. Hydrostatic weighing requires the subject to be completely immersed in water. This method of measurement is often impractical and costly. This method can be employed before and after a liposuction procedure, but would be impractical and costly when the goal is to monitor adipose tissue changes during the surgery. Additionally, the surgeon performing liposculpture and most surgical contouring procedures requires localized measurements. Maintenance of a sterile field is problematic with such a method.

Previous technologies also describe ultrasound transducers that require applying a fluid or gel to get effective acoustic coupling between the transducer and skin. This makes measurements messy and inconvenient for the subject.

A method and apparatus is needed to efficiently, accurately, conveniently and cost-effectively monitoring human adipose tissue (i.e., body fat). The present disclosure fulfills this need, and further provides related advantages.

### SUMMARY OF THE INVENTION

A system for accurately measuring, analyzing, and recording human body fat thickness is disclosed. The system can provide information about the health and fitness of a user. The system can use ultrasound signals transmitted and/or received by a hand held device that connects either through a cable (e.g., USB) or wireless technology (e.g., Bluetooth) to a computer that collects and analyzes the measurements to provide the user with information related to health and fitness. The data can be recorded to allow the user to track changes and monitor trends in their health and fitness. The application software can analyze the recorded data to provide the user with recommendations and health risks.

The system can accurately measure tissue layer thickness to monitor the effects of exercise or diet. The system can accurately measure percentage body fat and body density.

The system can accurately measure adipose tissue distribution and identify superficial adipose tissue and deep adipose tissue.

The system can have a remote control, a data processing unit, a handheld ultrasound transducer, a disposable sterile element to acoustically couple the transducer to skin and a monitor to display the information to the user.

The handheld ultrasound transducer can use a single or a plurality of ultrasound generating and detection elements to obtain an effective A-Scan (“Ultrasound in Medicine” Ed. F. A. Duck, A. C. Baker, H. C. Starritt (1997)) of the tissue structure directly below the transducer. The A-scan can detect strong reflections at the interface between the various layers i.e., skin, fat, muscle and bone. Strong ultrasound reflections occur at the interfaces due to impedance mismatches between the various materials. The A-scan signal can be analyzed by the control unit to determine the thickness of the various tissue layers (e.g., skin, fat, fat fascia, muscle). By making multiple measurements (e.g., chest, waist and thigh) a percent body fat for the whole body can be calculated. The device can be used to monitor fitness programs and diet.

The transducer can be connected by a wire or cable to the control unit. The wire or cable can be enclosed in a sterile sheath or bag. The transducer and control unit communicate through a wireless connection with the control unit (e.g., RF communication, such as bluetooth). The control unit and display can be far away from the sterile surgical field. The system can be without any wires between the transducer and the control unit, for example when RF communication is employed between the transducer and the control unit. The ultrasound transducer can be powered by a power source such as batteries or from the control unit via the wire or cable or wireless power transmission.

The remote control unit can acquire the data from the handheld transducer and analyze the data to produce a table of tissue thickness parameters for all the anatomical points. This data can be displayed in a tabulated list or a color-coded anatomical map that can be easily interpreted by the surgeon or user. The display can show the change in the fat layer thickness during the course of the liposuction procedure or otherwise over time. The user can control the display and function of the control unit through a keyboard/mouse interface or touch screen.

### BRIEF DESCRIPTION OF THE DRAWINGS

FIG. 1 is a cross-sectional view of a variation of the handheld ultrasound device with a disposable acoustic matching element.

FIG. 2 is a cross-sectional view of a variation of the handheld ultrasound device with a water compartment that can release a small amount of water to acoustically couple the ultrasound device to tissue.

FIG. 3 is a cross-sectional view of a variation of the handheld ultrasound device that has an integrated level and ruler.

FIG. 4 is a cross-sectional view of a variation of the handheld ultrasound device that has an integrated level and ruler.

FIG. 5 is a variation of the system for measuring body fat.

FIG. 6 illustrates a variation of a plot of the measured ultrasound signal on the thigh of a male.

FIG. 7 illustrates a variation of a plot of the measured ultrasound signal on the bicep of a male.

FIG. 8 illustrates a variation of the opening screen

FIG. 9 illustrates a variation of the Create New Client's Profile screen.

FIG. 10 illustrates a variation of the Open Existing Client screen.

FIG. 11 illustrates a variation of the BodyView screen for males.

FIG. 12 illustrates a variation of the BodyView screen for females.

FIG. 13 illustrates a variation of the Measure screen.

FIG. 14 illustrates a variation of the signal display screen.

FIG. 15 illustrates a variation of the My Health screen.

FIG. 16, not the invention, shows a cross sectional illustration of abdominal fat showing the two compartments of subcutaneous abdominal fat layer.

FIG. 17 shows a plot of the measured ultrasound signal of the abdomen of a male

FIG. 18 shows the Trends screen.

### DETAILED DESCRIPTION OF THE INVENTION

A system for evaluating health, wellness and fitness is disclosed. For example, the system can use an ultrasound transducer to accurately measure tissue layer thickness, such as fat thickness at a plurality of sites on a human or other animal body. The system can record the tissue layer thickness measurements for long term monitoring. The system can calculate the total body composition and/or health risks, for example using one or more of the tissue layer thickness measurements.

The system can be used to produce a map of the fat (or adipose) tissue thickness at key anatomical points. The map can be monitored and compared to track changes. The device can have a a remote control and data processing unit, a handheld ultrasound transducer, and a monitor or LCD to display the information to the user.

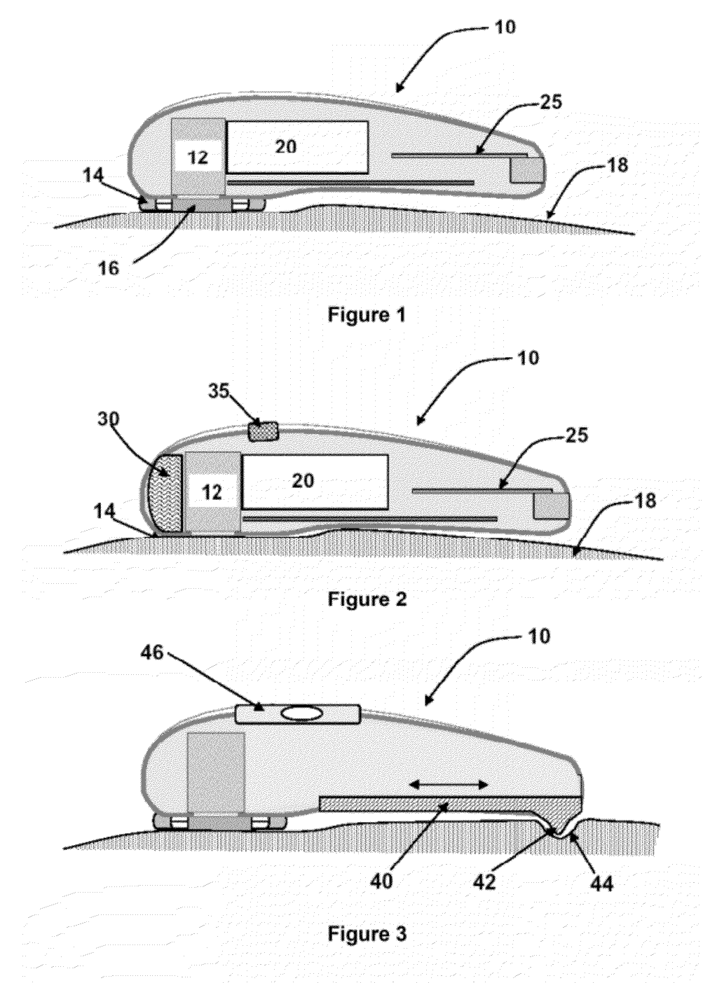

FIG. 1 shows a cross-sectional view of the handheld ultrasound measuring device 10. The device consists of an ultrasound transmitter and receiver 12. The transmitter and receiver can be a single element or two separate elements. The use of two separate elements reduces reflection artifacts and also allows imaging closer to the transmitter element. The ultrasound transmitter and detection element can be made of any piezoelectric material. Suitable materials include ceramics (usually lead zirconate titanate (PZT), or plastic (polyvinylidinedifluoride, PVDF). The operating frequency for adequate penetration and resolution in tissue is typically 500 kHz to 10 MHz. For additional information on transducer design and operation refer to “The Physics of Medical Imaging” Ed. Steve Webb (1988) incorporated herein by reference, and “Ultrasound in Medicine” Ed. F. A. Duck, A. C. Baker, H. C. Starritt (1997) incorporated herein by reference. See also U.S. Pat. No. 5,699,806, titled “Ultrasound System With Nonuniform Rotation Corrector” incorporated herein by reference.

In order to efficiently couple the ultrasound energy to the tissue it is important that a matching material is placed between the transducer and the tissue. This can be accomplished by applying a small amount of ultrasound coupling gel to the face of the transducer before applying it to tissue. Alternatively a disposable holder 14 connects to the device 10 to make acoustic contact between the transducer 12 and the matching material 16. The matching material is a high water fraction hydro gel or sol gel similar to that commonly used in electrocardiograms (ECG) electrodes or transcutaneous electric nerve stimulation (TENS) electrodes. The outside surface of the matching material 16 makes contact with the skin 18 and ensures good acoustic contact with minimal reflection at the skin interface. It is important that no air layer exists between the matching material 16 and the skin surface 18. An air layer produces a large reflection and significantly reduces the amount of ultrasound energy that is transmitted into the tissue. U.S. Pat. No. 6,792,301 (Munro et al.), incorporated herein by reference, and references therein describe a suitable material composition.

In order to reduce the risk of contamination a new disposable holder 14 can be used for each customer and visit. The use of a solid and adhesive matching material 16 avoids the need to apply acoustic gels or creams to the skin that need to be cleaned off after the procedure.

The device 10 can be powered by a battery 20 or external power cord (not shown). The measured signal can be transferred to a remote computer or microprocessor by wireless means 25 (e.g., Bluetooth, devices conforming to any wireless standard routinely used by computers e.g., IEEE 802.11, acoustic or optical) or cable (not shown). The device 10 can also be powered and also communicate to remote computer by a USB cable.

FIG. 2 shows that the device 10 can contain an integrated, refillable water compartment 30. The disposable holder is eliminated and acoustic coupling between the ultrasound transducer 12 and the skin 18 is made by a thin water layer. When making a measurement, the user presses button 35 that causes a small amount of water (1-2 drops) to be released near the surface of the ultrasound transducer 12. The water fills the gap between the transducer 12 and the skin 18 and allows efficient transmission into the tissue. The surface of the ultrasound transducer can be treated to be hydrophilic so that water will easily coat the surface. Instead of water a low viscosity oil or hydrogel could be used.

FIG. 3 shows that the device 10 can have an integrated ruler 40 (or measuring reference) that can be used to accurately position the transducer relative to a anatomical landmark. The ruler 40 can slide (left and right as shown) to allow the transducer to be placed at the desired distance from the bulbous tip 42. In addition, the device 10 can have an integrated level 46 to further allow the user to accurately set the orientation of the device. The level 46 can be a simple mechanical (e.g. water-bubble) level or an electronic IC based level with LED or LCD display. The ruler and level could be used to consistently make the measurement at the same anatomical position. This is important when monitoring changes over time. For example. by placing the bulbous tip 42 in the umbilicus 44 (belly button) it is possible to consistently make the tissue measurement at the same location.

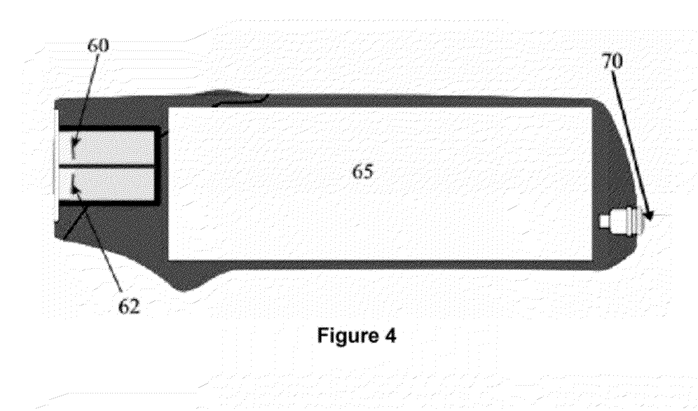

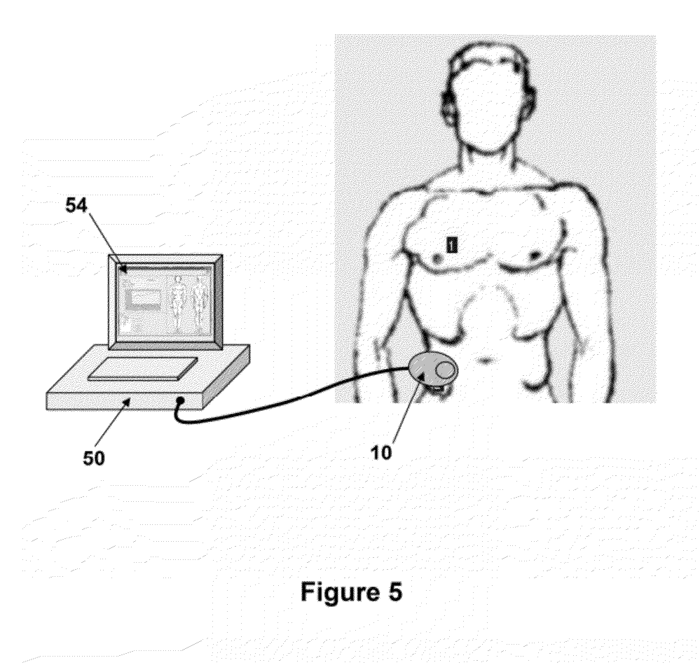

FIG. 4 shows that the device can have an ultrasound transmitter 60 and a separate receiver 62 integrated with the handheld device. A circuit board 65 drives the transmitter 60 and processes the received signal from the receiver 62 by amplifying it and filtering it before converting it to a digital signal that can be transmitted through the USB cable 70.

The system can have a hand held ultrasound transducer that can attach through a cable (e.g., USB) or wireless connection (e.g., Bluetooth) to a computer that can include a software program that can collect the recorded ultrasound signal. The software program can analyze the signal from each measurement point on the body and, using a minimum of one point, calculates the estimated total body fat. The program can also use multiple measurement points to increase total accuracy of the body fat measurement. Measured body fat percentage is used by the program to advise the user of fitness and relative risk of disease. Changes in the percentage of body fat are used to show the user the resulting modifications to the body shape.

FIG. 5 illustrates how the present invention can be used to measure the local tissue structure. The measuring device 10 is placed on the skin at a point of interest. When activated, an ultrasound signal is transmitted into the tissue and the return signal is collected. The collected signal'is then communicated by cable or by wireless means to the remote control unit 50. The control unit 50 displays the recorded waveforms and the calculated thickness of relevant layers on a monitor 54. In addition, the control unit 50 stores the waveforms and information about the location of the measurement so that the user can easily monitor changes over time. The control unit can be a portable computer, or PDA (e.g., HP Ipaq, Palm Pilot, etc.). In another embodiment, the device 10 is self contained and a small LCD display on the device 10 displays a summary of each measurement.

For the present invention, the operating frequency of the transducer will typically be in the range of 500 kHz to 10 MHz. The higher frequencies have higher spatial resolution but suffer from high tissue attenuation, which limits the thickness of tissue that can be measured. In addition, it is sometimes beneficial to operate the ultrasound transducer at two different frequencies. Since the scattered signal scales strongly with the ultrasound wavelength, the ratio of scattered signal at two frequencies can be used to determined tissue properties.

A curved transducer may be used to provide a weakly focused beam that measures properties over a less than 5 mm diameter region. A small diameter reduces the blurring of layer boundaries due to non-planar layer contours. The transducer is used to both generate the ultrasound pulse and measure the time history of the return acoustic signal. The collected time history signal is a measurement of the back-scattered signal as a function of depth averaged over the ultrasound beam area. The control electronics collect and digitize the signal for further display and analysis. For additional information on transducer design and operation refer to “The Physics of Medical Imaging” Ed. Steve Webb (1988), incorporated herein by reference, and “Ultrasound in Medicine” Ed. F. A. Duck, A. C. Baker, H. C. Starritt (1997), incorporated herein by reference. See also U.S. Pat. No. 5,699,806, titled: “Ultrasound System With Nonuniform Rotation Corrector”, incorporated herein by reference.

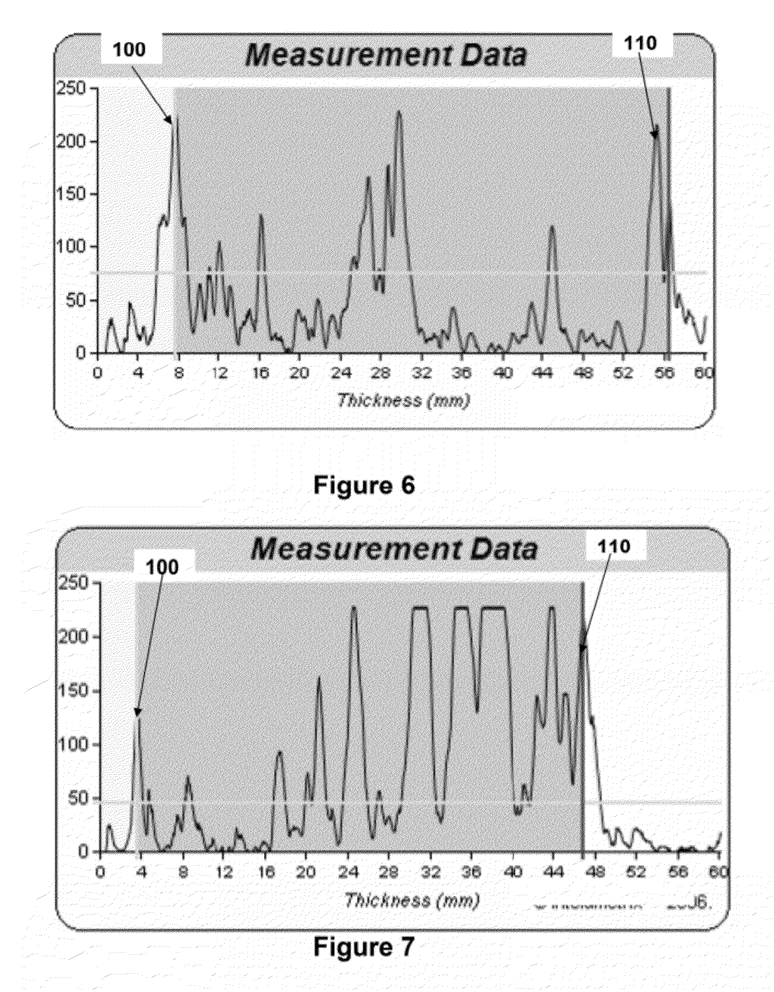

FIG. 6 shows a measured signal using the present invention on a male thigh. The signal peaks correspond to the interface between and fat and muscle 100 which is at approximately 8 mm. A strong signal 110 at approximately 55 mm is the reflection from the muscle bone interface. The muscle layer is located between 100 and 110 and is approximately 47 mm thick. Strong ultrasound reflections occur at the interfaces due to impedance mismatch between the various materials. The time history is converted to thickness by the software by using average sound speeds (c). For example, c˜1600 m/s for skin, 1400 m/s for fat, 1600 m/s for muscle, and 3500 m/s for bone (See “Ultrasound in Medicine” Ed. F. A. Duck, A. C. Baker, H. C. Starritt).

FIG. 7 shows a measured signal using the present invention on a male bicep muscle. The signal peaks correspond to the interface between fat and muscle 100 and muscle and bone 110. The adipose layer is located between skin surface and 110 and is approximately 3.2 mm thick. The muscle layer is located between 100 and 110 and is approximately 40.8 mm thick.

In order to accurately detect the interfaces the control software analyzes the signal and based on additional input information (e.g. measurement location, client weight, height, athletic type, age, and sex) determines the proper interface position. Strong signals are generally produced at each interface due to large difference in the acoustic impedance of the different tissue types. In addition, muscle tissue generally shows strong signal fluctuations and that information can be used to distinguish muscle from adipose tissue. Using client weight and height the body mass index can be calculated and using formulas that relate percentage body fat to body mass index (e.g. Deurenberg P, Yap M, van Staveren W A. Body mass index and percent body fat. A meta analysis among different ethnic groups. Int J Obes Relat Metab Disord 1998; 22:1164-1171.) the approximate thickness of adipose tissue can be calculated. Generally this estimated value can be 25%-50% too high for athletes. So in one version of the algorithm the user can input whether the client has an athletic build or not.

In normal use the measuring device would be applied at a single point or multiple key anatomical points. By making measurements at multiple sites (at least three) you can estimate the body density (D) and the percentage body fat (% BF). The most common sites used for these estimates are:

| | |
|---|---|
| TRICEPS | At the level of the mid-point between acromial process (boney tip of |
| | shoulder) and proximal end of the radius bone (elbow joint), on the |
| | posterior (back) surface of the arm. |
| BICEPS | The same level as for triceps, though on the anterior (front) surface |
| | of arm. |
| SUBSCAPULA | 2 cm below the lower angle of the scapula (bottom point of shoulder |
| | blade) on a line running laterally (away from the body) and |
| | downwards (at about 45 degrees). The fold is lifted in this direction. |
| AXILLA | The intersection of a horizontal line level with the bottom edge of |
| | the xiphoid process (lowest point of the breast bone), and a vertical |
| | line from the mid axilla (middle of armpit). |
| ILIAC CREST | The site immediately above the iliac crest (top of hip bone), at the |
| | mid-axillary line. |
| SUPRASPINALE | The intersection of a line joining the spinale (front part of iliac |
| | crest) and the anterior (front) part of the axilla (armpit), and a |
| | horizontal line at the level of the iliac crest. |
| ABDOMINAL | 5 cm adjacent to the umbilicus (belly-button). |
| FRONT THIGH | The mid-point of the anterior surface of the thigh, midway between |
| | patella (knee cap) and inguinal fold (crease at top of thigh). |
| MEDIAL CALF | The point of largest circumference on medial (inside) surface of the |
| | calf. |
| CHEST | Between the axilla and nipple as high as possible on the anterior |
| | axillary fold (males only). |

For example, by taking measurements at chest, abdomen, and thigh you can estimate the body density (D) and percentage body fat (% BF) with the following equations similar or equal to the following caliper equations for males and females, respectively.

For Males: D=1.10938−(0.0008267×sum of chest, abdominal, thigh)+(0.0000016×square of the sum of chest, abdominal, thigh)+(0.0002574×age). Equation is based on a sample of males aged 18-61 (Jackson, A. S. & Pollock, M. L. (1978) “Generalized equations for predicting body density of men”, British J of Nutrition, 40: p 497-504.).

D=1.1043−(0.001327×thigh)−(0.00131×subscapular), based on a sample aged 18-26. Sloan A W: “Estimation of body fat in young men”, J Appl. Physiol. (1967);23:p 311-315.

% BF=(0.1051×sum of triceps, subscapular, supraspinale, abdominal, thigh, calf)+2.585, based on a sample of college students. Yuhasz, M. S.: Physical Fitness Manual, London Ontario, University of Western Ontario, (1974).

For Females: D=1.0994921−(0.0009929×sum of triceps, suprailiac, thigh)+(0.0000023×square of the sum of triceps, suprailiac, thigh)−(0.0001392×age), based on a sample aged 18-55. Jackson, et al. (1980) “Generalized equations for predicting body density of women”, Medicine and Science in Sports and Exercise, 12:p 175-182.

D=1.0764−(0.0008×iliac crest)−(0.00088×tricep), based on a sample aged 17-25. Sloan, A. W., Burt A. J., Blyth C. S.: “Estimating body fat in young women”, J. Appl. Physiol. (1962);17:p 967-970.

% BF=(0.1548×sum of triceps, subscpular, supraspinale, abdominal, thigh, calf)+3.580, based on a sample of college students. Yuhasz, M. S.: Physical Fitness Manual, London Ontario, University of Western Ontario, (1974).

Although these equations refer to thickness measurements taken with calipers, they can also be applied when fat thickness measurements are made with the more accurate device disclosed herein. In addition, a wide variety of other equations exist that offer greater accuracy; however, some require additional information (e.g., accurate age, body type).

Software within the control unit can guide the user through the process of collecting measurements at the key anatomical sites and then display the calculated % body fat (% BF) and Body Density (D).

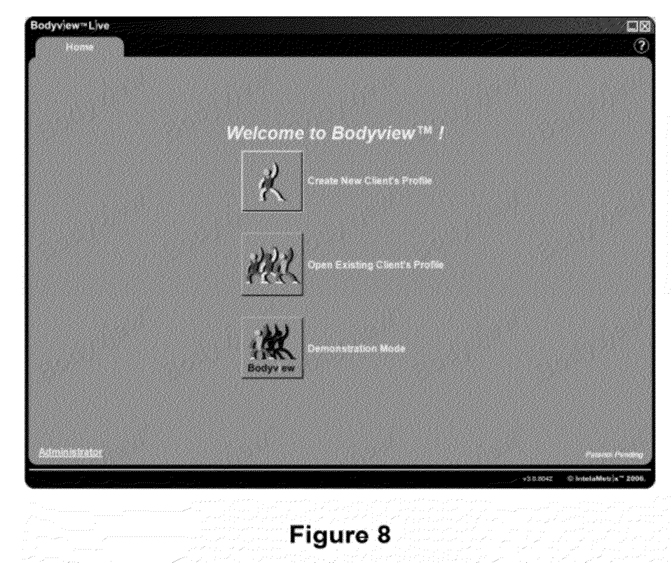

FIG. 8 shows a prototype of the present invention. A handheld ultrasound transducer 10 connected via an USB cable 20 to a laptop computer 50 running the body composition analysis software.

A software program (e.g., BodyView from IntelaMetrix, Livermore, Calif.) can control the ultrasound measurement device and display to the user with a wide variety of information tools, including body morphing extrapolated images and planning, fat thickness measurements, total body fat percentage measurement, trends and tracking, and health risk analyses. The program can run on a desktop computer, portable computer, or PDA device (e.g., HP IPAQ). The features and a sample of the screens displayed by the program are shown in FIGS. 8 through FIG. 14 and FIG. 17.

FIG. 8 shows an example of a Home Screen which allows the user to create a new client (or user), open the existing client data base or operate in a Demonstration mode where no data is recorded. Using option buttons the units of measure can be set to inches and pounds or centimeters and kilograms.

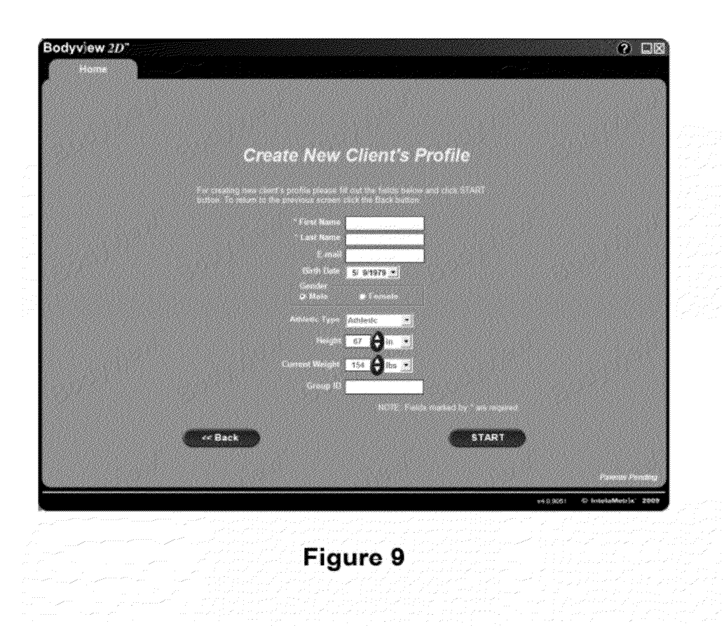

From the Home Screen the user can select to create a new client's profile. The Create New Client's Profile screen shown in FIG. 9 allows entry of the client's name, birth date, athletic type, height and weight.

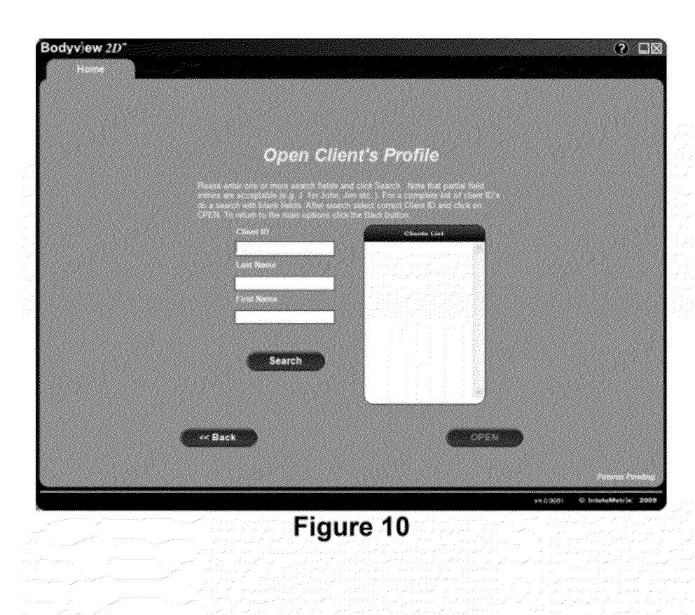

Also, from the Home Screen the user can open the existing client data base. The Open Existing Client screen (shown in FIG. 10) allows the user to retrieve previous measurements from the data base and look for trends.

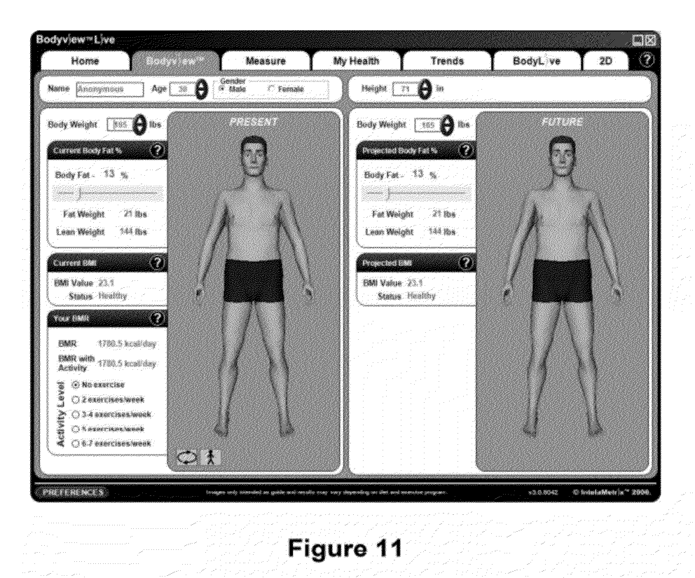

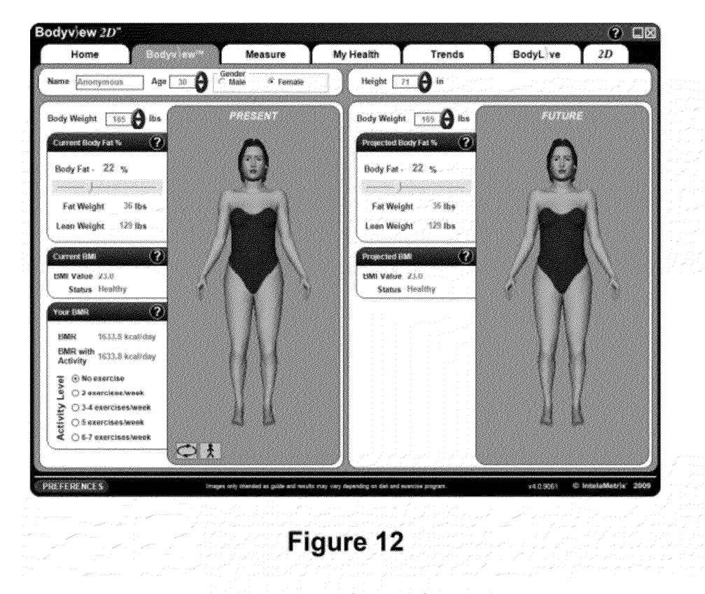

The BodyView screen (as shown in FIG. 11 for male and FIG. 12 for female) allows a client to adjust the percentage of body fat to get an approximate idea of how their body shape might change. The figures can be rotated to allow a view from all angles.

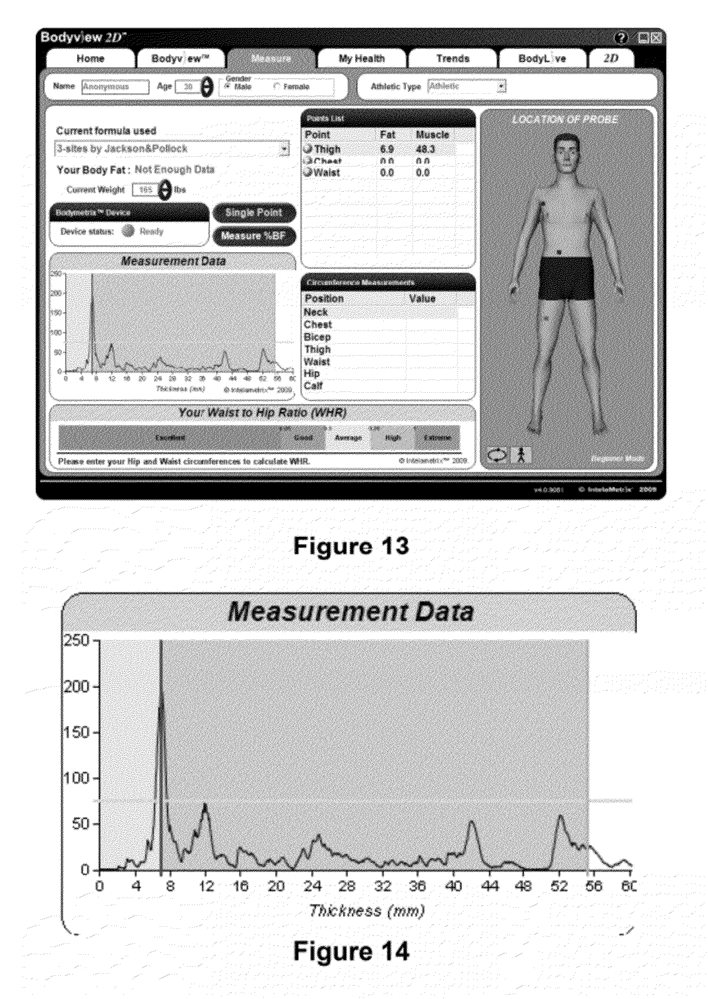

The Measure screen (FIG. 13) is used to control the measurement of fat thickness with the ultrasound transducer. From the Measure screen the user may select from a drop down menu a formula to calculate Body Fat. The formulas used are those known and accepted in the health and fitness fields (e.g., 2-site Sloan, 3-site and 7-site by Jackson & Pollock). When a measurement point is selected, the location on the pictured body is marked with a red cube (as shown on the thigh). The other measurement points are marked with blue cubes (shown elsewhere on the body other than the thigh). The user may add points by simply moving the cursor over the body picture and clicking on the desired locations. This feature allows a client to track the fat thickness in specific points of interest.

All measurements are taken from the Measure screen. To take a measurement, the user places the ultrasound device on the desired body point and presses the measure button, holding it down for approximately 1 second. When the button is released, the signal is analyzed and the estimated fat thickness and muscles thickness is displayed. This value is stored in the point list, and the user can move to the next measurement point. When all desired points are measured and recorded the body fat percentage is calculated and displayed.

The signal displayed in FIG. 14 shows a clear boundary between fat and muscle at approximately 6 mm. This is an example of the ultrasound measurement for a specific body point (male thigh).

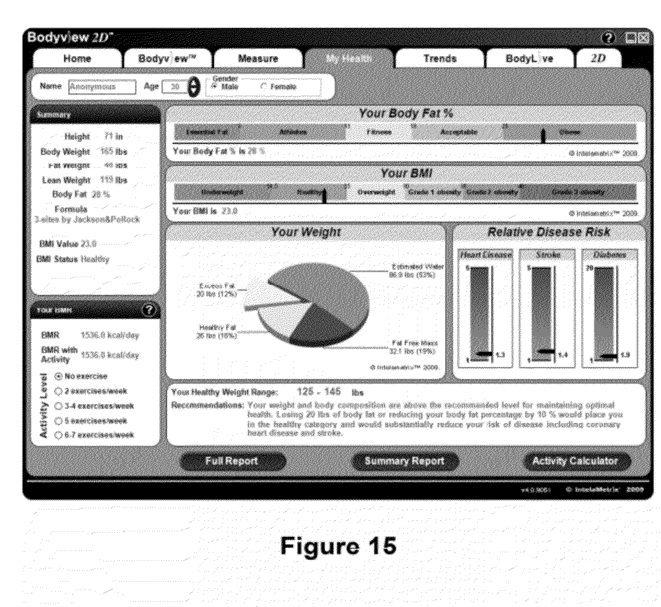

The My Health screen (FIG. 15) provides a summary of the user's present condition. This screen analyzes the information provided to give an overall picture of the user's total body composition and relative health risks. This information is provided as guidance. The user can print out a full report by clicking on the “Full Report” button or just the summary by clicking on the “Print Summary” button at the bottom of the page. The “Activity Calculator” button allows the user to calculate the number of calories burned by performing selected activities.

Relative Health Risk can be estimated from the Body Mass Index (BMI), the percentage body fat (% BF) or by analyzing the subcutaneous abdominal fat. Although BMI is a fast and convenient measurement its value in assessing disease state and health risk is less than optimum, particularly for muscular and athletic individuals. Interest in measurement of body composition has grown substantially since the early 1970's when the modern-day health and fitness movement began. Total percentage body fat (% BF) can now be measured by a variety of technologies and its use is becoming more widespread.

However, literature (e.g. Aroone L. J., Segal K. R. (2002b), Adiposity and Fat Distribution Outcome Measures: Assessment and Clinical Implications, Obesity Research 10(S1), 14S-21S) has consistently shown that adipose tissue distribution can be a more reliable predictor of chronic diseases then BMI or % BF. In particular, abdominal adipose tissue which can be divided into subcutaneous and visceral depots can be an accurate predictor of coronary disease (Ohlson L O, Larsson B, Svardsudd K, Welin L, Eriksson H, Wilhelmsen L, et al. (1985) The influence of body fat distribution on the incidence of diabetes mellitus. 13.5 years of follow-up of the participants in the study of men born in 1913. Diabetes 34,1055-8), and type 2 diabetes (Chan J M, Rimm E B, Colditz G A, Stampfer M J, Willett W C. (1994), Obesity, fat distribution, and weight gain as risk/actors for clinical diabetes in men. Diabetes Care 17,961-9, Despres J-P, Lemieux I, Prud'homme D. (2001), Treatment of obesity: need to focus on high risk abdominally obese patients. B M J 322, 716-20).

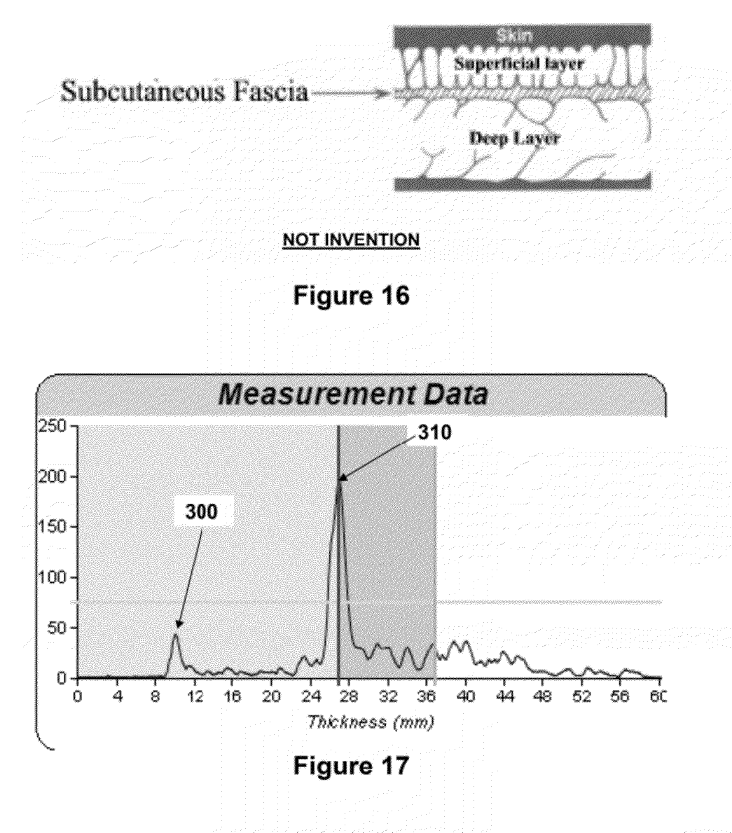

The subcutaneous adipose depots can be further divided into superficial adipose tissue (SAT) and deep adipose tissue (DAT) compartments (see FIG. 16) which are separated by subcutaneous fascia. The rationale for this division initially came from animal studies which indicate that lipids are depleted and deposited at a faster rate into the deep layer of the subcutaneous tissue then the superficial layer. This suggests that the superficial layer acts as a thermal insulation or storage layer whereas the deep layer functions as a metabolically active tissue (Carey, G. B. (1997), The swine as a model for studying exercise induced changes in lipid metabolism. Medicine and Science in Sports and Exercise 29, 1437-43). These animal studies were confirmed by Monzon et al (Monzon, J. R., Basile, R., Heneghan, S. Udupi, V., and Green, A. (2002), Lipolysis in adipocitves isolated from deep and superficial subcutaneous adipose tissue. Obesity Research 10, 266-9) who reported that lipolytic activity was higher in adipocytes isolated from DAT compared with adipoctyes isolated from SAT. DAT, but NOT SAT has been found to be strongly related to insulin resistance in a cohort of lean and obese men and women.

Therefore beyond BMI, % BF and Waist to Flip Ratio, a direct measurement of the SAT and DAT in the abdominal region offers an improved health risk index that can be used to identify populations with higher risk for cardiovascular disease, diabetes, and stroke.

The system can accurately measure the SAT and DAT layers as shown in FIG. 17. The fascia signal 300 representing the subcutaneous fascia between the SAT and the DAT is shown by ultrasound peak at approximately 10 mm. The muscle interface signal 310 representing the change from the DAT and the muscle is shown by the ultrasound peak. at approximately 27 mm. For this male the SAT is approximately 10 mm thick (i.e., the difference between 0 mm and the depth of the fascia signal at 10 mm) and the DAT is approximately 17 mm thick (i.e., the difference between the fascia signal 300 at 10 mm and the muscle interface signal 310 at approximately 27 mm). The muscle depth and other layer thicknesses can also be calculated.

The computer in the system can automatically determine the fascia signal 300 and the muscle interface signal 310, for example by threshold analysis of the signal. The y-axis of FIG. 17 is shown in arbitrary units of signal strength for illustrative purposes only. For example, the computer can scan the signal starting from 0 depth and progressing deeper to find the first signal peak above 25 arbitrary units to determine the fascia signal 300. The computer can then continue to scan the signal deeper than the fascia signal 300 to find the next signal peak above 25 arbitrary units to determine the muscle interface signal 310. The computer can also filter the signal for width of the peaks and adjust the filter used to search for the fascia signal 300 and the muscle interface signal 310 based on BMI, age, location on the body of the signal (e.g., chest, thigh), and body type (e.g., elite athlete, average, non-athletic).

The signals shown in FIG. 6 and FIG. 7 are examples of ultrasound measurements made at different anatomical points.

The software can calculate a ratio of SAT thickness to DAT thickness (i.e., “SAT:DAT ratio”) to determine health risks The system can compare the SAT:DAT ratio, age, body type, BMI, body fat percentage, gender, personal behavior (e.g., smoking, diet), family health history, or combinations thereof of the present subject with a database or reference chart to determine the relative health risks for subjects having the same or similar characteristics. The software can present the health risk factors to the user via any of the screens, such as the Trends Screen or in the Relative Disease Risk window of the My Health Screen where risks for Heart Disease, Stroke, Diabetes, Cancer, or combinations thereof.

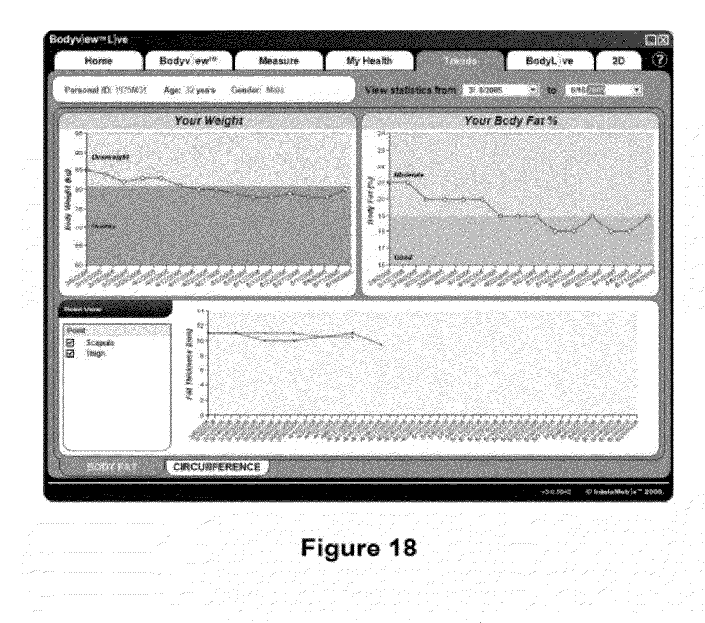

The Trends screen shown in FIG. 18 tracks a user's body composition over time. The Trends screen allows the user to monitor the changes or trends in BMI, Body Fat percentage or fat thickness at selected points. The patient can set goals in the software. The Trends screen can illustrate how the user is performing compared to interpolated points toward the user's goal.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

The foregoing description is presented for purposes of illustration and description and is not intended to be exhaustive or to limit the invention to the precise variations disclosed. Many modifications and variations are possible in light of the above teaching. The variations were chosen and described to explain the principles of the disclosure and its practical application to thereby enable others skilled in the art to best use the disclosure in variations and with various modifications suited to the particular use contemplated, and to make and use the disclosure with any combinations of features and elements described herein.

We claim:

1. A method of presenting information regarding the health of a subject comprising:
transmitting an ultrasound signal into the user;
receiving reflections of the signal from the user; and
analyzing the reflections of the signal, wherein analyzing comprises determining the thicknesses of the superficial adipose tissue (SAT) and the deep adipose tissue (DAT).

2. The method of claim 1, further comprising retrieving health risks for the subject by referencing a database with the thicknesses of the SAT and the DAT.

3. The method of claim 1, wherein transmitting comprises transmitting at a location on the subject, and wherein analyzing comprises using at least one parameter that is specific to the subject.

4. The method of claim 3, wherein the at least one parameter is selected from the group consisting of: age, height, weight, athletic type, gender, and location of said skin portion.

5. The method of claim 1, further comprising:
applying at least one ultrasound transducer to the surface of a skin portion of a subject under test; and wherein transmitting comprises transmitting ultrasound pulses from the transducer into a skin portion of the subject, wherein an interface between a first layer and a second layer beneath the skin portion reflect a portion of the ultrasound pulses to produce a return signal; and
wherein receiving comprises receiving the return signal.

6. The method of claim 5, wherein the first layer comprises an adipose tissue layer, and wherein the second layer comprises a muscle layer.

7. The method of claim 5, wherein the first layer comprises an SAT layer, and wherein the second layer comprises a DAT layer.

8. The method of claim 1, further comprising producing a map of fat thickness.

9. The method of claim 1, wherein transmitting, receiving and analyzing are performed at a plurality of anatomical points to determine adipose tissue thickness at each anatomical point, the method further comprising calculating a percentage of body fat of the subject by using the plurality of adipose tissue thicknesses, and at least one parameter that is specific to the subject wherein at least one parameter is selected from the group consisting of: age, height, weight, athletic type, gender, and location of said anatomical points.

10. A method of presenting information regarding the health of a subject comprising:
transmitting an ultrasound signal into the user with an ultrasound transducer;
receiving reflections of the signal from the user with the ultrasound transducer; and
converting the reflections of the signal to a representative signal that is representative of the reflections of the ultrasound-signal, wherein the converting is performed at least in part by the ultrasound transducer.
transmitting the representative signal from the transducer to a computer system;
analyzing the representative signal, wherein analyzing the representative signal comprises determining with the computer system the ratio of the thickness of the SAT to the thickness of the DAT.

11. The method of claim 10, wherein analyzing comprises determining with the computer system a location of the subcutaneous fascia between the SAT and the DAT.

12. The method of claim 10, wherein analyzing further comprises comparing the ratio of the thickness of the SAT to the thickness of the DAT and at least one additional parameter of the group consisting of: age, body type, body mass index (BMI), body fat percentage, gender, smoking habits, diet, and family health history.

13. The method of claim 10, wherein analyzing further comprises comparing the ratio of the thickness of the SAT to the thickness of the DAT and body fat percentage.

14. The method of claim 10, wherein analyzing further comprises identifying the location of at least one major tissue interface from the group consisting of skin-subcutaneous fat layer interface, subcutaneous fat layer-muscle interface, and muscle-bone interface.

15. A method of presenting information regarding the health of a subject comprising:
transmitting an ultrasound signal into the user with an ultrasound transducer;
receiving reflections of the signal from the user with the ultrasound transducer; and
converting the reflections of the signal to a representative signal that is representative of the reflections of the ultrasound signal, wherein the converting is performed at least in part by the ultrasound transducer.
transmitting the representative signal from the transducer to a computer system;
analyzing the representative signal, wherein analyzing the representative signal comprises determining with the computer system the thickness of the DAT.

16. The method of claim 15, wherein analyzing comprises determining with the computer system a location of the subcutaneous fascia between the SAT and the DAT.

17. The method of claim 15, wherein analyzing further comprises comparing the thickness of the DAT and at least one additional parameter of the group consisting of thickness of the SAT, age, body type, BMI, body fat percentage, gender, smoking habits, diet, and family health history.

18. The method of claim 15, wherein analyzing further comprises comparing the ratio of the thickness of the DAT and body fat percentage.

19. The method of claim 15, wherein analyzing further comprises identifying the location of at least one major tissue interface from the group consisting of skin-subcutaneous fat layer interface, subcutaneous fat layer-muscle interface, and muscle-bone interface.
